(19) United States
(12) Patent Application Publication
(10) Pub. No.: US 2002/0022619 A1
(43) Pub. Date: Feb. 21, 2002

(54) Tricyclic fused pyridine and pyrimidine derivatives as CRF antagonists

(76) Inventor: Rajagopal Bakthavatachalam, Wilmington, DE (US)

Correspondence Address: BRISTOL-MYERS SQUIBB PHARMA COMPANY, PATENT DEPARTMENT, P.O. BOX 4000, PRINCETON, NJ, 08543-4000, US

(21) Appl. No.: 09/845,867

(22) Filed: Apr. 30, 2001

Related U.S. Application Data
(60) Provisional application No. 60/200,982, filed on May 1, 2000.

(51) Int. Cl. C07D 487/16; A61K 31/5517

(52) U.S. Cl. 514/220; 540/554; 540/549

(57) ABSTRACT

The present invention relates to tricyclic fused pyrimidine and pyridine derivatives having the following general formula:

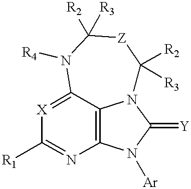

Said compounds bind to the CRF receptor, and are thus useful in the treatment of anxiety, depression and other related disorders.

### FIELD OF THE INVENTION

The present invention relates to a group of tricyclic fused pyrimidine and pyridine derivatives which bind to the CRF receptor, and are thus useful in the treatment of anxiety, depression and other related disorders.

### BACKGROUND OF THE INVENTION

Corticotropin releasing factor (herein referred to as CRF), a 41 amino acid peptide, is the primary physiological regulator of proopiomelanocortin (POMC)-derived derived peptide secretion from the anterior pituitary gland [J. Rivier et al., Proc. Nat. Acad. Sci. (USA) 80:4851 (1983); W. Vale et al., Science 213:1394 (1981)]. In addition to its endocrine role at the pituitary gland, immunohistochemical localization of CRF has demonstrated that the hormone has a broad extrahypothalamic distribution in the central nervous system and produces a wide spectrum of autonomic, electrophysiological and behavioral effects consistent with a neurotransmitter or neuromodulator role in brain [W. Vale et al., Rec. Prog. Horm. Res. 39:245 (1983); G. F. Koob, Persp. Behav. Med. 2:39 (1985); E. B. De Souza et al., J. Neurosci. 5:3189 (1985)]. There is also evidence that CRF plays a significant role in integrating the response of the immune system to physiological, psychological, and immunological stressors [J. E. Blalock, Physiological Reviews 69:1 (1989); J. E. Morley, Life Sci. 41:527 (1987)].

Clinical data provide evidence that CRF has a role in psychiatric disorders and neurological diseases including depression, anxiety-related disorders and feeding disorders. A role for CRF has also been postulated in the etiology and pathophysiology of Alzheimer's disease, Parkinson's disease, Huntington's disease, progressive supranuclear palsy and amyotrophic lateral sclerosis as they relate to the dysfunction of CRF neurons in the central nervous system [for review see E. B. De Souza, Hosp. Practice 23:59 (1988)].

In affective disorder, or major depression, the concentration of CRF is significantly increased in the cerebral spinal fluid (CSF) of drug-free individuals [C. B. Nemeroff et al., Science 226:1342 (1984); C. M. Banki et al., Am. J. Psychiatry 144:873 (1987); R. D. France et al., Biol. Psychiatry 28:86 (1988); M. Arato et al., Biol Psychiatry 25:355 (1989)]. Furthermore, the density of CRF receptors is significantly decreased in the frontal cortex of suicide victims, consistent with a hypersecretion of CRF [C. B. Nemeroff et al., Arch. Gen. Psychiatry 45:577 (1988)]. In addition, there is a blunted adrenocorticotropin (ACTH) response to CRF (i.v. administered) observed in depressed patients [P. W. Gold et al., Am J. Psychiatry 141:619 (1984); F. Holsboer et al., Psychoneuroendocrinology 9:147 (1984); F. W. Gold et al., New Eng. J. Med. 314:1129 (1986)]. Preclinical studies in rats and non-human primates provide additional support for the hypothesis that hypersecretion of CRF may be involved in the symptoms seen in human depression [R. M. Sapolsky, Arch. Gen. Psychiatry 46:1047 (1989)]. There is preliminary evidence that tricyclic antidepressants can alter CRF levels and thus modulate the numbers of CRF receptors in brain [Grigoriadis et al., Neuropsychopharnacology 2:53 (1989)].

It has also been postulated that CRF has a role in the etiology of anxiety-related disorders. CRF produces anxiogenic effects in animals and interactions between benzodiazepine/non-benzodiazepine anxiolytics and CRF have been demonstrated in a variety of behavioral anxiety models [D. R. Britton et al., Life Sci. 31:363 (1982); C. W. Berridge and A. J. Dunn Regul. Peptides 16:83 (1986)]. Preliminary studies using the putative CRF receptor antagonist a-helical ovine CRF (9-41) in a variety of behavioral paradigms demonstrate that the antagonist produces “anxiolytic-like” effects that are qualitatively similar to the benzodiazepines [C. W. Berridge and A. J. Dunn Horm. Behav. 21:393 (1987), Brain Research Reviews 15:71 (1990)].

Neurochemical, endocrine and receptor binding studies have all demonstrated interactions between CRF and benzodiazepine anxiolytics, providing further evidence for the involvement of CRF in these disorders. Chlordiazepoxide attenuates the “anxiogenic” effects of CRF in both the conflict test [K. T. Britton et al., Psychopharmacology 86:170 (1985); K. T. Britton et al., Psychopharmacology 94:306 (1988)] and in the acoustic startle test [N. R. Swerdlow et al., Psychopharmacology 88:147 (1986)] in rats. The benzodiazepine receptor antagonist (Ro15-1788), which was without behavioral activity alone in the operant conflict test, reversed the effects of CRF in a dose-dependent manner while the benzodiazepine inverse agonist (FG7142) enhanced the actions of CRF [K. T. Britton et al., Psychopharmacology 94:306 (1988)].

It has been further postulated that CRF has a role in immunological, cardiovascular or heart-related diseases such as hypertension, tachycardia and congestive heart failure, stroke, osteoporosis, premature birth, psychosocial dwarfism, stress-induced fever, ulcer, diarrhea, post-operative ileus and colonic hypersensitivity associated with psychopathological disturbance and stress.

The mechanisms and sites of action through which the standard anxiolytics and antidepressants produce their therapeutic effects remain to be elucidated. It has been hypothesized however, that they are involved in the suppression of the CRF hypersecretion that is observed in these disorders. Of particular interest is that preliminary studies examining the effects of a CRF receptor antagonist (a-helical CRF9-41) in a variety of behavioral paradigms have demonstrated that the CRF antagonist produces “anxiolytic-like” effects qualitatively similar to the benzodiazepines [for review see G. F. Koob and K. T. Britton, In: Corticotropin-Releasing Factor: Basic and Clinical Studies of a Neuropeptide, E. B. De Souza and C. B. Nemeroff eds., CRC Press p221 (1990)].

The following publications each describe CRF antagonist compounds; however, none disclose the compounds provided herein: WO95/10506; WO99/51608; WO97/35539; WO99/01439; WO97/44308; WO97/35846; WO98/03510; WO99/11643; PCT/US99/18707; WO99/01454; and, WO00/01675.

### SUMMARY OF THE INVENTION

This invention provides a compound of formula I:

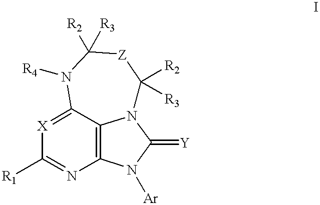

wherein: X is N or CR¹; Y is O, S or CH₂; Z is CH₂, C═O, C═S, NR¹ or a single bond; Ar is phenyl, naphthyl, pyridyl, pyrimidinyl, triazinyl, furanyl, quinolinyl, isoquinolinyl, thienyl, imidazolyl, thiazolyl, indolyl, pyrrolyl, oxazolyl, benzofuranyl, benzothienyl, benzthiazolyl, isoxazolyl or pyrazolyl, each optionally substituted with 1 to 4 R⁵ groups; R¹ is H, C₁-C₄ alkyl, C₂-C₄ alkenyl, C₂-C₄ alkynyl, halogen, CN, C₁-C₄ haloalkyl, —NR⁹R¹⁰, NR⁹COR¹⁰, —OR¹¹, SH or —S(O)ₙR¹²; R² is H, C₁-C₄ alkyl, allyl, C₃-C₆ cycloalkyl, halogen, CN, —NR⁶R⁷, NR⁹COR¹⁰, C₁-C₄ haloalkyl, or —S(O)ₙR¹²; R³ is H, C₁-C₄ alkyl, allyl, or propargyl, where C₁-C₄ alkyl is optionally substituted with C₃-C₆ cycloalkyl, halogen, CN, —NR⁶R⁷, —OR⁷, —S(O)ₙR¹² or —CO₂R⁷; R⁴ is NR⁶R⁷, —OR⁷, C₁-C₁₀ alkyl, C₂-C₁₀ alkenyl, C₂-C₁₀ alkynyl, C₃-C₈ cycloalkyl or C₄-C₁₂ cycloalkylalkyl each optionally substituted with 1 to 3 substituents independently selected at each occurrence from C₁-C₆ alkyl, C₃-C₆ cycloalkyl, halo, C₁-C₄ haloalkyl, cyano, SH, —S(O)ₙR¹³, —CO₂R⁷,—NR⁸COR⁷, —NR⁸CONR⁶R⁷, —NR⁸CO₂R¹³, —aryl and heteroaryl, where the aryl or heteroaryl is optionally substituted with 1 to 3 substituents independently selected at each occurrence from C₁-C₄ alkyl, halo, cyano, —OR⁷, —S(O)ₙR⁷, —CO₂R⁷, —NR⁸COR⁷, —NR⁸CONR⁶R⁷, —NR⁸CO₂R⁷, and —NR⁶R⁷; R⁵ is C₁-C₁₀ alkyl, C₂-C₁₀ alkenyl, C₂-C₁₀ alkynyl, C₃-C₆ cycloalkyl, C₄-C₁₂ cycloalkylalkyl, —NO₂, halo, —CN, C₁-C₄ haloalkyl, —NR⁶R⁷, COR⁷ —OR⁷, —CONR⁶R⁷, —CO(NOR⁹)R⁷, C₂R⁷, or —S(O)ₙR⁷, where C₁-C₁₀ alkyl, C₂-C₁₀ alkenyl, C₂-C₁₀ alkynyl, C₃-C₆ cycloalkyl and C₄-C₁₂ cycloalkylalkyl are optionally substituted with 1 to 3 substituents independently selected at each occurrence from C₁-C₄ alkyl, —NO₂, halo, —CN, —NR⁶R⁷, COR⁷ —OR⁷, —CONR⁶R⁷, CO₂R⁷, —CO(NOR⁹)R⁷, or —S(O)ₙR⁷; R⁶ and R⁷ are independently at each occurrence H, C₁-C₄ alkyl, C₁-C₄ haloalkyl, C₂-C₈ alkoxyalkyl, C₃-C₆ cycloalkyl, C₄-C₁₂ cycloalkylalkyl, aryl, aryl(C₁-C₄alkyl)-, heteroaryl or heteroaryl(C₁-C₄ alkyl)-; or NR⁶R⁷ is piperidine, pyrrolidine, piperazine, N-methylpiperazine, morpholine or thiomorpholine; R⁸ is H or C₁-C₄ alkyl; R⁹ and R¹⁰ are independently selected from H or C₁-C₄ alkyl, C₃-C₆ cycloalkyl; R¹¹ is H, C₁-C₄ alkyl, C₁-C₄ haloalkyl, C₃-C₆ cycloalkyl; R¹² is C₁-C₄ alkyl or C₁-C₄ haloalkyl; and, R¹³ is C₁-C₄ alkyl, C₁-C₄ haloalkyl, C₂-C₈ alkoxyalkyl, C₃-C₆ cycloalkyl, C₄-C₁₂ cycloalkylalkyl, aryl (aryl is phenyl or naphthyl optionally substituted with 1 to 3 substituents independently selected at each occurrence from C₁-C₄ alkyl, halo, cyano, —OR⁷, SH, —S(O)ₙR¹², —CO₂R⁸, —NR⁸COR⁷, —NR⁸CONR⁶R⁷, —NR⁸CO₂R¹², and —NR⁶R⁷), aryl(C₁-C₄ alkyl)-, heteroaryl or heteroaryl(C₁-C₄ alkyl)-, or NR⁶R⁷. Preferred embodiments of this invention are described hereinbelow.

This invention also provides pharmaceutical compositions containing such compounds, as well as methods of treating anxiety, depression and other CRF-mediated disorders using said compositions.

### DETAILED DESCRIPTION OF THE INVENTION

This invention provides a compound of formula I:

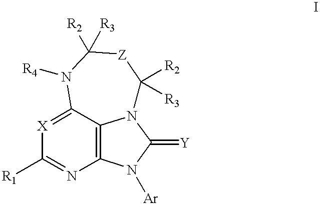

wherein: X is N or CR¹; Y is O, S or CH₂; Z is CH₂, C═O, C═S, NR¹ or a single bond; Ar is phenyl, naphthyl, pyridyl, pyrimidinyl, triazinyl, furanyl, quinolinyl, isoquinolinyl, thienyl, imidazolyl, thiazolyl, indolyl, pyrrolyl, oxazolyl, benzofuranyl, benzothienyl, benzthiazolyl, isoxazolyl or pyrazolyl, each optionally substituted with 1 to 4 R⁵ groups; heteroaryl is pyridyl, pyrimidinyl, triazinyl, furanyl, quinolinyl, isoquinolinyl, thienyl, imidazolyl, thiazolyl, indolyl, pyrrolyl, oxazolyl, benzofuranyl, benzothienyl, benzthiazolyl, isoxazolyl or pyrazolyl optionally substituted with 1 to 3 substituents independently selected at each occurrence from C₁-C₄ alkyl, halo, cyano, —OR⁷, SH, —S(O)ₙR¹², —CO₂R⁸, —NR⁸COR⁷, —NR⁸CONR⁶R⁷, —NR⁸CO₂R¹², and —NR⁶R⁷); n is independently at each occurrence 0,1 or 2; R¹ is H, C₁-C₄ alkyl, C₂-C₄ alkenyl, C₂-C₄ alkynyl, halogen, CN, C₁-C₄ haloalkyl, —NR⁹R¹⁰, NR⁹COR¹⁰, —OR¹¹, SH or —S(O)ₙR¹²; R² is H. C₁-C₄ alkyl, allyl, C₃-C₆ cycloalkyl, halogen, CN, —NR⁶R⁷, NR⁹COR¹⁰, C₁-C₄ haloalkyl, or —S(O)ₙR¹²; R³ is H, C₁-C₄ alkyl, allyl, or propargyl, where C₁-C₄ alkyl is optionally substituted with C₃-C₆ cycloalkyl, halogen, CN, —NR⁶R⁷, —OR⁷, —S(O)ₙR¹² or —CO₂R⁷; R⁴ is NR⁶R⁷, —OR⁷, C₁-C₁₀ alkyl, C₂-C₁₀ alkenyl, C₂-C₁₀ alkynyl, C₃-C₈ cycloalkyl or C₄-C₁₂ cycloalkylalkyl each optionally substituted with 1 to 3 substituents independently selected at each occurrence from C₁-C₆ alkyl, C₃-C₆ cycloalkyl, halo, C₁-C₄ haloalkyl, cyano, SH, —S(O)ₙR¹³ —CO₂R⁷, —NR⁸COR⁷, —NR⁸CONR⁶R⁷, —NR⁸CO₂R¹³, —aryl and heteroaryl, where the aryl or heteroaryl is optionally substituted with 1 to 3 substituents independently selected at each occurrence from C₁-C₄ alkyl, halo, cyano, —OR⁷, —S(O)ₙR⁷, —C₂R⁷, —NR⁸COR⁷—NR⁸CONR⁶R⁷, —NR⁸CO₂R⁷, and —NR⁶R⁷; R⁵ is C₁-C₁₀ alkyl, C₂-C₁₀ alkenyl, C₂-C₁₀ alkynyl, C₃-C₆ cycloalkyl, C₄-C₁₂ cycloalkylalkyl, —NO₂, halo, —CN, C₁-C₄ haloalkyl, —NR⁶R⁷, COR⁷ —OR⁷, —CONR⁶R⁷, —CO(NOR⁹)R⁷, C₂R⁷, or —S(O)ₙR⁷, where C₁-C₁₀ alkyl, C₂-C₁₀ alkenyl, C₂-C₁₀ alkynyl, C₃-C₆ cycloalkyl and C₄-C₁₂ cycloalkylalkyl are optionally substituted with 1 to 3 substituents independently selected at each occurrence from C₁-C₄ alkyl, —NO₂, halo, —CN, —NR⁶R⁷, COR⁷ —OR⁷, —CONR⁶R⁷, CO₂R⁷, —CO(NOR⁹)R⁷, or —S(O)ₙR⁷; R⁶ and R⁷ are independently at each occurrence H, C₁-C₄ alkyl, C₁-C₄ haloalkyl, C₂-C₈ alkoxyalkyl, C₃-C₆ cycloalkyl, C₄-C₁₂ cycloalkylalkyl, aryl, aryl(C₁-C₄alkyl)-, heteroaryl or heteroaryl(C₁-C₄ alkyl)-; or NR⁶R⁷ is piperidine, pyrrolidine, piperazine, N-methylpiperazine, morpholine or thiomorpholine; R⁸ is H or C₁-C₄ alkyl; R⁹ and R¹⁰ are independently selected from H or C₁-C₄ alkyl, C₃-C₆ cycloalkyl; R¹¹ is H, C₁-C₄ alkyl, C₁-C₄ haloalkyl, C₃-C₆ cycloalkyl; R¹² is C₁-C₄ alkyl or C₁-C₄ haloalkyl; and, R¹³ is C₁-C₄ alkyl, C₁-C₄ haloalkyl, C₂-C₈ alkoxyalkyl, C₃-C₆ cycloalkyl, C₄-C₁₂ cycloalkylalkyl, aryl (aryl is phenyl or naphthyl optionally substituted with 1 to 3 substituents independently selected at each occurrence from C₁-C₄ alkyl, halo, cyano, —OR⁷, SH, —S(O)ₙR¹², —CO₂R⁸, —NR⁸COR⁷, —NR⁸CONR⁶R⁷, —NR⁸CO₂R¹², and —NR⁶R⁷), aryl(C₁-C₄ alkyl)-, heteroaryl or heteroaryl(C₁-C₄ alkyl)-, or NR⁶R^{7.}

Preferably, X is N, Y is O, Z is CH₂, R¹ is CH₃, R₂ and R₃ are H at each occurrence thereof, Ar is 2-bromo-4-isopropyl phenyl and R⁴ is C₂H₅, C₄H₉, C₅H₁₁, CH(C₂H₅)C₂H₅, CH₂-C₃ cyclopropyl or —CH₂-C₆H₅.

As used herein, the following terms have the following terms have the following meanings. “Alkyl” means saturated hydrocarbon chains, branched or unbranched, having the specified number of carbon atoms. “Alkenyl” means hydrocarbon chains of either a straight or branched configuration and one or more unsaturated carbon-carbon bonds, which may occur in any stable point along the chain, such as ethenyl, propenyl, and the like. “Alkynyl” means hydrocarbon chains of either a straight or branched configuration and one or more triple carbon-carbon bonds, which may occur in any stable point along the chain, such as ethynyl, propynyl and the like. “Alkoxy” means an alkyl group of indicated number of carbon atoms attached through an oxygen bridge. “Cycloalkyl” means saturated ring groups, including mono-,bi- or polycyclic ring systems, such as cyclopropyl, cyclobutyl, cyclopentyl, cyclohexyl, and so forth. “Halo” or “halogen” means fluoro, chloro, bromo, and iodo. “Haloalkyl” means both branched and straight-chain alkyls having the specified number of carbon atoms, substituted with 1 or more halogens. “Haloalkoxy” means an alkoxy group substituted by at least one halogen atom.

Substituent groupings, e.g., C₁₋₄ alkyl, are known, and are hereby stated, to include each of their individual substituent members, e.g., C₁ alkyl, C₂ alkyl, C₃ alkyl and C₄ alkyl. “Substituted” means that one or more hydrogen on the designated atom is replaced with a selection from the indicated group, provided that the designated atom's normal valency is not exceeded, and that the substitution results in a stable compound. “Unsubstituted” atoms bear all of the hydrogen atoms dictated by their valency. When a substituent is keto, then 2 hydrogens on the atom are replaced. Combinations of substituents and/or variables are permissible only if such combinations result in stable compounds; by “stable compound” or “stable structure” is meant a compound that is sufficiently robust to survive isolation to a useful degree of purity from a reaction mixture, and formulation into an efficacious therapeutic agent.

Pharmaceutically acceptable salts of compounds of this invention are also provided herein. The phrase “pharmaceutically acceptable” is employed to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. “Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines, or alkali or organic salts of acidic residues such as carboxylic acids. Pharmaceutically acceptable salts include the conventional non-toxic salts or the quaternary ammonium salts of the parent compound formed, for example, from non-toxic inorganic or organic acids. Such conventional nontoxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

Pharmaceutically acceptable salt forms of compounds provided herein are synthesized from the parent compound which contains a basic or acidic moiety by conventional chemical methods. Generally, such salts are, for example, prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is hereby incorporated by reference.

6,7,8,9-Tetrahydro-2H-2,3,5,6,9a-pentaaza-benzo [cd]azulen-1-one compounds (1) of the present invention may be obtained by following the steps outlined in Scheme 1:

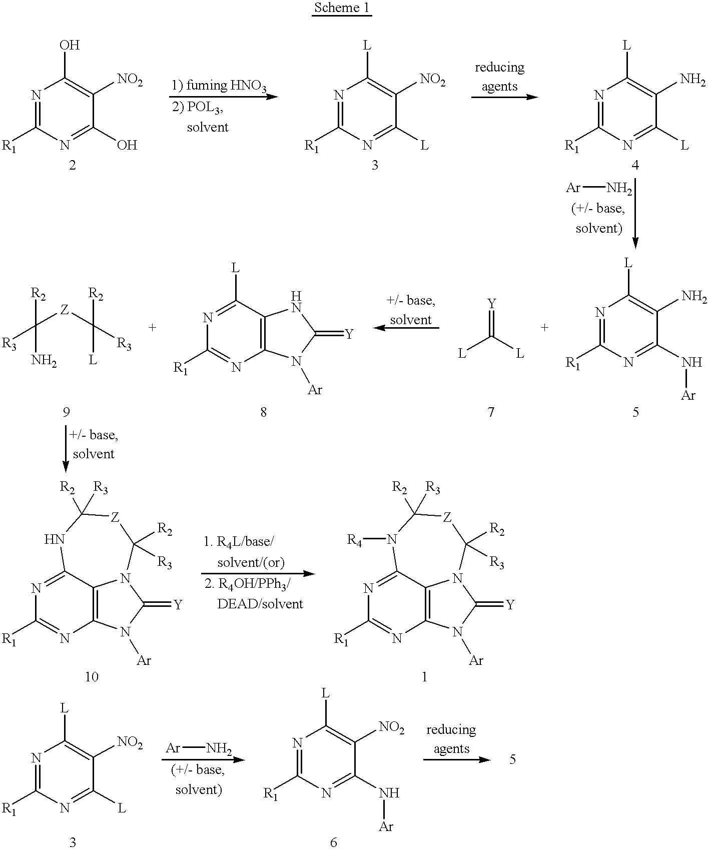

Compounds of the formula (2) may be nitrated using nitrating agents but not limited to fuming nitric acid and then converted to compounds of formula (3) by treatment with phosphorus oxyhalides, phosphorus halides, alkyl sulfonyl halides, aryl sufonyl halides (L=halogen, sulfonates). Compounds of the formula (3), may be reduced to amino derivatives of formula (4) using methods known in literature. Anilinopyrimidine derivative (5) can be obtained by treatment of compound (4) with aniline in the presence or absence of a base in solvents such as aliphatic alcohols or an inert solvent at temperatures ranging from −20° C. to 200° C. Bases may include, but are not limited to, alkali metal carbonates, alkali metal bicarbonates, trialkyl amines (preferably N,N-di-isopropyl-N-ethyl amine) or aromatic amines (preferably pyridine). Alternatively, compounds of formula (5) may be obtained from compounds of formula (6) as shown in the Scheme 1. Compounds of formula (5) may be converted to compound of formula (8) by treatment with reagents of the formula (7), wherein L=leaving group (halogen, imidazole) and Y═O, S. Compounds of formula (10) may be obtained by treatment of compound of formula (8) with compound of formula (9) in the presence or absence of a base in solvents such as aliphatic alcohols or an inert solvent at temperatures ranging from −20° C. to 200° C. Compounds of the formula (10) may be alkylated by treatment with R₄L (L=leaving group) in the presence of base in an inert solvent at temperatures ranging from −20° C. to 200° C. Bases may include, but are not limited to, alkali metal hydrides (preferably sodium hydride), alkaline earth metal hydrides, alkali metal dialkylamides (preferably lithium di-isopropylamide) and alkali metal bis(trialkylsilyl)amides (preferably sodium bis(trimethylsilyl)amide). Inert solvents may include, but are not limited to, lower alkanenitriles (1 to 6 carbons, preferably acetonitrile), dialkyl ethers (preferably diethyl ether), cyclic ethers (preferably tetrahydrofuran or 1,4-dioxane), N,N-dialkylformamides (preferably dimethylformamide), N,N-dialkylacetamides (preferably dimethylacetamide), cyclic amides (preferably N-methylpyrrolidin-2-one), dialkylsulfoxides (preferably dimethylsulfoxide), aromatic hydrocarbons (preferably benzene or toluene) or haloalkanes of 1 to 10 carbons and 1 to 10 halogens (preferably dichloromethane). Alternatively, intermediates (10) may then be reacted with alcohols R₄OH in the presence of phosphines R^{a}₃P (where R^{a} is lower alkyl, phenyl or substituted phenyl or furyl) and an azodicarboxylate ester R^{b}O₂CN═NCO₂R^{b} (where R^{b} is lower alkyl) in an inert solvent at temperatures ranging from −80° C. to 150° C. Inert solvents may include, but are not limited to, polyethers (preferably 1,2-dimethoxyethane), dialkyl ethers (preferably diethyl ether), cyclic ethers (preferably tetrahydrofuran or 1,4-dioxane) or aromatic hydrocarbons (preferably benzene or toluene). The choices of phosphine, solvent or azodicarboxylate ester are known to those skilled in the art as described by 0. Mitsunobu (Synthesis, 1, 1981).

The following examples are provided to describe the invention in further detail. These examples, which set forth the best mode presently contemplated for carrying out the invention, are intended to illustrate and not to limit the invention.

### EXAMPLES

Analytical data were recorded for the compounds described below using the following general procedures. Proton NMR spectra were recorded on a Varian FT-NMR (300 MHz); chemical shifts were recorded in ppm (δ) from an internal tetramethysilane standard in deuterochloroform or deuterodimethylsulfoxide as specified below. Mass spectra (MS) or high resolution mass spectra (HRMS) were recorded on a Finnegan MAT 8230 spectrometer (using chemical ionization (CI) with NH₃ as the carrier gas or gas chromatography (GC) as specified below) or a Hewlett Packard 5988A model spectrometer. Melting points were recorded on a Buchi Model 510 melting point apparatus and are uncorrected. Boiling points are uncorrected. All pH determinations during workup were made with indicator paper.

Reagents were purchased from commercial sources and, where necessary, purified prior to use according to the general procedures outlined by D. Perrin and W. L. F. Armarego, Purification of Laboratory Chemicals, 3rd ed., (New York: Pergamon Press, 1988). Chromatography (thin layer (TLC) or preparative) was performed on silica gel using the solvent systems indicated below. For mixed solvent systems, the volume ratios are given. Otherwise, parts and percentages are by weight.

Synthesis of 2-(2-Bromo-4-isopropyl-phenyl)-6-ethyl-4-methyl-6,7,8,9-tetrahydro-2H-2,3,5,6,9a-pentaaza-benzo[cd]azulen-1-one

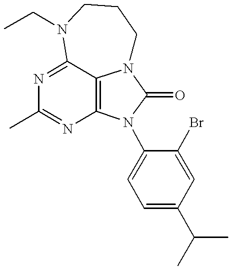

Part A: N-[4-{2-bromo-4-(1-methylethyl)phenyl}]-6-chloro-2-methyl pyrimidin-4,5-diamine:5-Amino-4,6-dichloro-2-methylpyrimidine (28.5 g, 0.16 mol) and 2-bromo-4-isopropylaniline (34.24 g, 0.16 mol) in 2-ethoxyethanol (100 mL) were refluxed at 135° C. for 30 h. After cooling the reaction mixture, the solvent was removed in vacuo and the residue taken up into dichloromethane; the organic phase was washed with water, dried over anhydrous magnesium sulfate and filtered. Solvent removal gave an oil that was purified by flash chromatography (silica gel) using methanol/CH₂Cl₂ (1:100) to yield the desired product as a cream colored solid (32.1 g, 56% yield, mp 144.5-146° C.).

Part B: 8-Oxo-Purine: The diamine from Part A of Example 1 (3.55 g, 10.0 mmol) was dissolved in dry toluene (20.0 mL) under nitrogen. To this mixture was added 20% COCl₂ (20 mL, 39 mmole, 3.9 equiv.) and refluxed for 90 mins. TLC (1:50 MeOH/CH₂Cl₂) revealed a new spot (Rf=0.24). The reaction mixture was cooled to room temp, neutralized with dilute NaHCO₃, extracted with EtOAc (3×15 mL), dried (MgSO₄) and concentrated in vacuum to white solid (3.6 g, 93% yield, mp 226-228° C.).

Part C: 2-(2-Bromo-4-isopropyl-phenyl)-4-methyl-6,7,8,9-tetrahydro-2H-2,3,5,6,9a-pentaaza-benzo[c,d]azulen-1-one: The product from Part B (1.35 g, 3.5 mmol) was dissolved in absolute ethanol (20 mL) and treated with triethylamine (1.4 g, 14.0 mmol, 4.0 equiv) and 3-chloropropylamine hydrochloride (0.48 g, 3.7 mmol, 1.05 equiv.). The resulting mixture was refluxed under nitrogen for 48 h. Solvent from the reaction mixture was removed under vacuum, extracted with EtOAc (3×50 mL), washed with brine, dried (MgSO₄) and concentrated in vacuum to afford residue. The residue was purified by flash column chromatography on a silica gel using 0.5% MeOH in CH₂Cl₂ to afford white solid (0.75 g, mp 264-265° C.). Anal calcd. for C₁₈H₂₀BrN₅O: C, 53.74; H, 5.01; N, 17.41. Found: C, 53.63; H, 4.95; N, 17.27.

Part D: Title Commpound: The amine from Part C of Example 1 (210.0 mg, 0.5 mmol) was dissolved in dry DMF (5.0 mL) under nitrogen. To this mixture was added 60% NaH (40 mg, 1.0 mmol, 2 equiv.) and stirred at room temperature for 10 mins. EtI (excess) was added to the mixture and stirred at room temperature for 3 days. TLC (1:50 MeOH/CH₂Cl₂) revealed a new spot (Rf=0.44). The reaction mixture was quenched with water (50.0 mL), stirred the mixture for 10 mins., extracted with EtOAc (3×15 mL), dried (MgSO₄) and concentrated in vacuum to afford yellow oil. The residue was purified by flash column chromatography on a silica gel using CH₂Cl₂ to afford white solid (120 mg, mp 74-76° C.). Anal calcd. for C₂₀H₂₄BrN₅O: C, 55.82; H, 5.62; N, 16.27. Found: C, 55.70; H, 5.59; N, 16.13.

### 2-(2-Bromo-4-isopropyl-phenyl)-6-cyclopropylmethyl-4-methyl-6,7,8,9-tetrahydro-2H-2,3,5,6,9a-pentaaza-benzo[cd]azulen-1-one

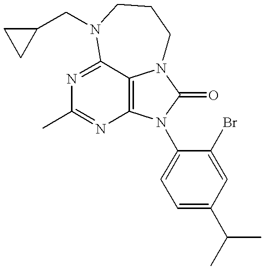

The amine from Part C of Example 1 (250.0 mg, 0.62 mmol) was dissolved in dry DMF (5.0 mL) under nitrogen. To this mixture was added 60% NaH (50 mg, 1.24 mmol, 2 equiv.) and stirred at room temperature for 10 mins. 1-Bromomethylcyclopropane (excess) was added to the mixture and stirred at room temperature for 2 days. TLC (1:50 MeOH/CH₂Cl₂) revealed a new spot (Rf=0.44). The reaction mixture was quenched with water (50.0 mL), stirred the mixture for 10 mins., extracted with EtOAc (3×15 mL), dried (MgSO₄) and concentrated in vacuum to afford yellow oil. The residue was purified by flash column chromatography on a silica gel using CH₂Cl₂ to afford white solid (150 mg, mp 89-90° C.). Anal calcd. for C₂₂H₂₆BrN₅O: C, 57.90; H, 5.74; N, 15.35. Found: C, 57.79; H, 5.74; N, 15.13.

### 2-(2-Bromo-4-isopropyl-phenyl)-6-butyl-4-methyl-6,7,8,9-tetrahydro-2H-2,3,5,6,9a-pentaaza-benzo[cd]azulen-1-one

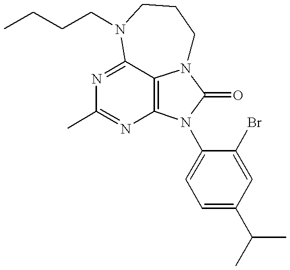

The amine from Part C of Example 1 (100.0 mg, 0.25 mmol) was dissolved in dry DMF (5.0 mL) under nitrogen. To this mixture was added 60% NaH (20 mg, 0.3 mmol, 1.2 equiv.) and stirred at room temperature for 10 mins. 1-Bromobutane (41 mg, 0.3 mmol, 1.2 equiv.) was added to the mixture and stirred at room temperature for 24 hour. TLC (1:10 MeOH/CH₂Cl₂) revealed a new spot (Rf=0.9). The reaction mixture was quenched with water (50.0 mL), stirred the mixture for 10 mins., extracted with EtOAc (3×15 mL), dried (MgSO₄) and concentrated in vacuum to afford yellow oil. The residue was purified by flash column chromatography on a silica gel using 1% MeOH in CH₂Cl₂ to afford white amorphous solid (75 mg, yield 65%). Anal calcd. for C₂₂H₂₈BrN₅O: C, 57.64; H, 6.17; N, 15.28. Found: C, 57.57; H, 6.15; N, 15.21.

### 2-(2-Bromo-4-isopropyl-phenyl)-4-methyl-6-pentyl-6,7,8,9-tetrahydro-2H-2,3,5,6,9a-pentaaza-benzo[cd]azulen-1-one

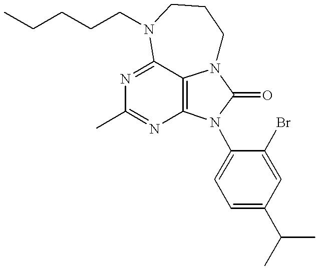

The amine from Part C of Example 1 (100.0 mg, 0.25 mmol) was dissolved in dry DMF (5.0 mL) under nitrogen. To this mixture was added 60% NaH (20 mg, 0.3 mmol, 1.2 equiv.) and stirred at room temperature for 10 mins. 1-Bromopentane (45 mg, 0.3 mmol, 1.2 equiv.) was added to the mixture and stirred at room temperature for 24 hour. TLC (1:10 MeOH/CH₂Cl₂) revealed a new spot (Rf=0.94). The reaction mixture was quenched with water (50.0 mL), stirred the mixture for 10 mins., extracted with EtOAc (3×15 mL), dried (MgSO₄) and concentrated in vacuum to afford yellow oil. The residue was purified by flash column chromatography on a silica gel using 1% MeOH in CH₂Cl₂ to afford white amorphous solid (65 mg, yield 55%). Anal calcd. for C₂₃H₃₀BrN₅O: C, 58.47; H, 6.40; N, 14.82. Found: C, 58.46; H, 6.40; N, 14.72.

### 2-(2-Bromo-4-isopropyl-phenyl)-6-(1-ethyl-propyl)-4-methyl-6,7,8,9-tetrahydro-2H-2,3,5,6,9a-pentaaza-benzo[cd]azulen-1-one

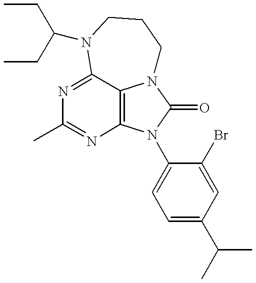

The amine from Part C of Example 1 (300.0 mg, 0.75 mmol), PPh₃ (0.24 g, 0.90 mmol, 1.2 equiv.) and 3-pentanol (0.1 mL, 0.90 mmol, 1.2 equiv) were dissolved in 15 mL of anhydrous THF (added 4A molecular sieves to the reaction mixture) and cooled to 0° C. Then diisopropylazodicarboxylate (0.182 g, 0.90 mmol, 1.2 equiv.) was added using a syringe. The reaction mixture was stirred at 0° C. for 2 h and then at room temp. for 2 days. Some insoluble material also separated from the reaction mixture. TLC (30:70 EtOAc/hexane) revealed a new spot (Rf=0.57) along with some unreacted starting material (Rf=0.36). Mass spec (NH₃-CI revealed presence of anticipated product along with another material with M+H=279 (may be Ph₃P═O). Some unreacted starting material was also noticed. The solvent was stripped off in vacuo and the residue was purified by flash column chromatography (30:70 EtOAc+hexane) to afford 0.26 g of white solid after crystallization from pentane. NMR indicated desired product along with some unidentified impurities but TLC showed single spot under UV (non UV active impurities). As a result, dissolved the sample in diethyl ether (25 mL) and washed with 1.0 M HCl and the organic layer was dried and upon concentration white fluffy solid separated from the solution was filtered and dried (150 mg, 40% yield, mp 149-150° C.).

### 2-(2-Bromo-4-isopropyl-phenyl)-4-methyl-6-styryl-6,7,8,9-tetrahydro-2H-2,3,5,6,9a-pentaaza-benzo[cd]azulen-1-one

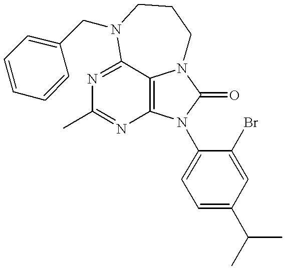

The amine from Part C of Example 1 (100.0 mg, 0.25 mmol) was dissolved in dry DMF (5.0 mL) under nitrogen. To this mixture was added 60% NaH (20 mg, 0.3 mmol, 1.2 equiv.) and stirred at room temperature for 10 mins. Benzyl bromide (51 mg, 0.3 mmol, 1.2 equiv.) was added to the mixture and stirred at room temperature for 24 hour. TLC (1:10 MeOH/CH₂Cl₂) revealed a new spot (Rf=0.94). The reaction mixture was quenched with water (50.0 mL), stirred the mixture for 10 mins., extracted with EtOAc (3×15 mL), dried (MgSO₄) and concentrated in vacuum to afford yellow oil. The residue was purified by flash column chromatography on a silica gel using 1% MEOH in CH₂Cl₂ to afford white amorphous solid (75 mg, yield 61%

This invention also provides a pharmaceutical composition comprising a pharmaceutically acceptable carrier and a therapeutically effective amount of a compound provided herein. “Pharmaceutically acceptable carriers” are media generally accepted in the art for the delivery of biologically active agents to animals, in particular, mammals. Such media are formulated according to a number of factors well within the purview of those of ordinary skill in the art to determine and account for. These include, without limitation: the type and nature of the active agent being formulated; the subject to which the agent-containing composition is to be administered; the intended route of administration of the composition; and, the therapeutic indication being targeted.

Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, well known to those of ordinary skill in the art.

Pharmaceutical compositions suitable for parenteral administration include various aqueous media such as aqueous dextrose and saline solutions; glycol solutions are also useful carriers, and preferably contain a water soluble salt of the active ingredient, suitable stabilizing agents, and if necessary, buffer substances. Antioxidizing agents, such as sodium bisulfite, sodium sulfite, or ascorbic acid, either alone or in combination, are suitable stabilizing agents; also used are citric acid and its salts, and EDTA. In addition, parenteral solutions can contain preservatives such as benzalkonium chloride, methyl- or propyl-paraben, and chlorobutanol.

Alternatively, compositions can be administered orally in solid dosage forms, such as capsules, tablets and powders; or in liquid forms such as elixirs, syrups, and/or suspensions. Gelatin capsules can be used to contain the active ingredient and a suitable carrier such as but not limited to lactose, starch, magnesium stearate, stearic acid, or cellulose derivatives. Similar diluents can be used to make compressed tablets. Both tablets and capsules can be manufactured as sustained release products to provide for continuous release of medication over a period of time. Compressed tablets can be sugar-coated or film-coated to mask any unpleasant taste, or used to protect the active ingredients from the atmosphere, or to allow selective disintegration of the tablet in the gastrointestinal tract.

Descriptions of suitable pharmaceutically acceptable carriers, and factors involved in their selection, are found in a variety of readily available sources, e.g., Remington's Pharmaceutical Sciences, 17th ed., Mack Publishing Company, Easton, Pa., 1985, the contents of which are incorporated herein by reference.

Compounds provided herein are antagonists of receptors for corticotropin releasing factor (“CRF”), a 41 amino acid peptide that is the primary physiological regulator of proopiomelanocortin (POMC)-derived peptide secretion from the anterior pituitary gland [J. Rivier et al., Proc. Nat. Acad. Sci. (USA) 80:4851 (1983); W. Vale et al., Science 213:1394 (1981)]. Immunohistochemical localization of CRF has also demonstrated that CRF has a broad extrahypothalamic distribution in the central nervous system and produces a wide spectrum of autonomic, electrophysiological and behavioral effects consistent with a neurotransmitter or neuromodulator role in brain [W. Vale et al., Rec. Prog. Horm. Res. 39:245 (1983); G. F. Koob, Persp. Behav. Med. 2:39 (1985); E. B. De Souza et al., J. Neurosci. 5:3189 (1985)]. There is also evidence that CRF plays a significant role in integrating the response of the immune system to physiological, psychological, and immunological stressors [J. E. Blalock, Physiological Reviews 69:1 (1989); J. E. Morley, Life Sci. 41:527 (1987)].

CRF concentrations have been found to be significantly increased in the cerebral spinal fluid (CSF) of drug-free individuals afflicted with affective disorder or depression [C. B. Nemeroff et al., Science 226:1342 (1984); C. M. Banki et al., Am. J. Psychiatry 144:873 (1987); R. D. France et al., Biol. Psychiatry 28:86 (1988); M. Arato et al., Biol Psychiatry 25:355 (1989)]. Furthermore, the density of CRF receptors is significantly decreased in the frontal cortex of suicide victims, consistent with a hypersecretion of CRF [C. B. Nemeroff et al., Arch. Gen. Psychiatry 45:577 (1988)]. Moreover, there is a blunted adrenocorticotropin (ACTH) response to CRF (i.v. administered) observed in depressed patients [P. W. Gold et al., Am J. Psychiatry 141:619 (1984); F. Holsboer et al., Psychoneuroendocrinology 9:147 (1984); P. W. Gold et al., New Eng. J. Med. 314:1129 (1986)].

CRF produces anxiogenic effects in animals. Moreover, interactions between benzodiazepine/non-benzodiazepine anxiolytics and CRF have been demonstrated in a variety of behavioral anxiety models [D. R. Britton et al., Life Sci. 31:363 (1982); C. W. Berridge and A. J. Dunn Regul. Peptides 16:83 (1986)]. Preliminary studies using the putative CRF receptor antagonist alpha-helical ovine CRF (9-41) in a variety of behavioral paradigms demonstrate that the antagonist produces “anxiolytic-like” effects that are qualitatively similar to the benzodiazepines [C. W. Berridge and A. J. Dunn Horm. Behav. 21:393 (1987), Brain Research Reviews 15:71 (1990)]. Neurochemical, endocrine and receptor binding studies have all demonstrated interactions between CRF and benzodiazepine anxiolytics, providing further evidence for the involvement of CRF in these disorders. Chlordiazepoxide attenuates the “anxiogenic” effects of CRF in both the conflict test [K. T. Britton et al., Psychopharmacology 86:170 (1985); K. T. Britton et al., Psychopharmacology 94:306 (1988)] and in the acoustic startle test [N. R. Swerdlow et al., Psychopharmacology 88:147 (1986)] in rats. The benzodiazepine receptor antagonist (Ro15-1788), which was without behavioral activity alone in the operant conflict test, reversed the effects of CRF in a dose-dependent manner while the benzodiazepine inverse agonist (FG7142) enhanced the actions of CRF [K. T. Britton et al., Psychopharmacology 94:306 (1988)]. The contents of the above-cited documents are incorporated herein by reference.

Thus, compounds provided herein which, because of their antagonism of CRF receptors, alleviate the effects of CRF overexpression are expected to be useful in treating these and other disorders. Such treatable disorders include, for example and without limitation: affective disorder, anxiety, depression, headache, irritable bowel syndrome, post-traumatic stress disorder, supranuclear palsy, immune suppression, Alzheimer's disease, gastrointestinal diseases, anorexia nervosa or other feeding disorder, drug addiction, drug or alcohol withdrawal symptoms, inflammatory diseases, cardiovascular or heart-related diseases, fertility problems, human immunodeficiency virus infections, hemorrhagic stress, obesity, infertility, head and spinal cord traumas, epilepsy, stroke, ulcers, amyotrophic lateral sclerosis and hypoglycemia.

This invention thus further provides a method of treating a subject afflicted with a disorder characterized by CRF overexpression, such as those described hereinabove, which comprises administering to the subject a pharmaceutical composition provided herein. Such compositions generally comprise a therapeutically effective amount of a compound provided herein, that is, an amount effective to ameliorate, lessen or inhibit disorders characterized by CRF overexpression. “Therapeutically effective amounts” typically comprise from about 0.1 to about 1000 mg of the compound per kg of body weight of the subject to which the composition is administered. Therapeutically effective amounts can be administered according to any dosing regimen satisfactory to those of ordinary skill in the art.

What is claimed is:

1. A compound of formula I: 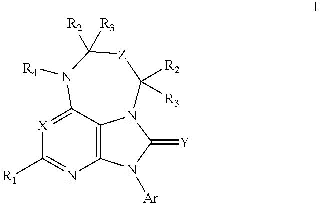
wherein:
X is N or CR¹
Y is O, S or CH₂
Z is CH₂, C═O, C═S, NR¹ or a single bond
Ar is phenyl, naphthyl, pyridyl, pyrimidinyl, triazinyl, furanyl, quinolinyl, isoquinolinyl, thienyl, imidazolyl, thiazolyl, indolyl, pyrrolyl, oxazolyl, benzofuranyl, benzothienyl, benzthiazolyl, isoxazolyl or pyrazolyl, each optionally substituted with 1 to 4 R⁵ groups;
heteroaryl is pyridyl, pyrimidinyl, triazinyl, furanyl, quinolinyl, isoquinolinyl, thienyl, imidazolyl, thiazolyl, indolyl, pyrrolyl, oxazolyl, benzofuranyl, benzothienyl, benzthiazolyl, isoxazolyl or pyrazolyl optionally substituted with 1 to 3 substituents independently selected at each occurrence from C₁-C₄ alkyl, halo, cyano, —OR⁷, SH, —S(O)ₙR¹², —CO₂R⁸, —NR⁸COR⁷, —NR⁸CONR⁶R⁷, —NR⁸CO₂R¹², and —NR⁶R⁷)
n is independently at each occurrence 0,1 or 2;
R¹ is H, C₁-C₄ alkyl, C₂-C₄ alkenyl, C₂-C₄ alkynyl, halogen, CN, C₁-C₄ haloalkyl, —NR⁹R¹⁰, NR⁹COR¹⁰, —OR¹¹, SH or —S(O)ₙR¹²;
R² is H, C₁-C₄ alkyl, allyl, C₃-C₆ cycloalkyl, halogen, CN, —NR⁶R⁷, NR⁹COR¹⁰, C₁-C₄ haloalkyl, or —S(O)ₙR¹²;
R³ is H, C₁-C₄ alkyl, allyl, or propargyl, where C₁-C₄ alkyl is optionally substituted with C₃-C₆ cycloalkyl, halogen, CN, —NR⁶R⁷, —OR⁷, —S(O)ₙR¹² or —CO₂R⁷;
R⁴ is NR⁶R⁷, —OR⁷, C₁-C₁₀ alkyl, C₂-C₁₀ alkenyl, C₂-C₁₀ alkynyl, C₃-C₈ cycloalkyl or C₄-C₁₂ cycloalkylalkyl each optionally substituted with 1 to 3 substituents independently selected at each occurrence from C₁-C₆ alkyl, C₃-C₆ cycloalkyl, halo, C₁-C₄ haloalkyl, cyano, SH, —S(O)ₙR¹³, —CO₂R⁷, —NR⁸COR⁷, —NR⁸CONR⁶R⁷, —NR⁸CO₂R¹³, -aryl and heteroaryl, where the aryl or heteroaryl is optionally substituted with 1 to 3 substituents independently selected at each occurrence from C₁-C₄ alkyl, halo, cyano, —OR⁷, —S(O)ₙR⁷, —CO₂R⁷, —NR⁸COR⁷, —NR⁸CONR⁶R⁷, —NR⁸CO₂R⁷, and —NR⁶R⁷;
R⁵ is C₁-C₁₀ alkyl, C₂-C₁₀ alkenyl, C₂-C₁₀ alkynyl, C₃-C₆ cycloalkyl, C₄-C₁₂ cycloalkylalkyl, —NO₂, halo, —CN, C₁-C₄ haloalkyl, —NR⁶R⁷, COR⁷ —OR⁷, —CONR⁶R⁷, —CO(NOR⁹)R⁷, CO₂R⁷, or —S(O)ₙR⁷, where C₁-C₁₀ alkyl, C₂-C₁₀ alkenyl, C₂-C₁₀ alkynyl, C₃-C₆ cycloalkyl and C₄-C₁₂ cycloalkylalkyl are optionally substituted with 1 to 3 substituents independently selected at each occurrence from C₁-C₄ alkyl, —NO₂, halo, —CN, —NR⁶R⁷, COR⁷—OR⁷, —CONR⁶R⁷, CO₂R⁷, —CO(NOR⁹)R⁷, or —S(O)ₙR⁷;
R⁶ and R⁷ are independently at each occurrence H, C₁-C₄ alkyl, C₁-C₄ haloalkyl, C₂-C₈ alkoxyalkyl, C₃-C₆ cycloalkyl, C₄-C₁₂ cycloalkylalkyl, aryl, aryl(C₁-C₄alkyl)-, heteroaryl or heteroaryl (C₁-C₄ alkyl)-; or NR⁶R⁷ is piperidine, pyrrolidine, piperazine, N-methylpiperazine, morpholine or thiomorpholine;
R⁸ is H or C₁-C₄ alkyl;
R⁹ and R¹⁰ are independently selected from H or C₁-C₄ alkyl, C₃-C₆ cycloalkyl;
R¹¹ is H, C₁-C₄ alkyl, C₁-C₄ haloalkyl, C₃-C₆ cycloalkyl;
R¹² is C₁-C₄ alkyl or C₁-C₄ haloalkyl; and,
R¹³ is C₁-C₄ alkyl, C₁-C₄ haloalkyl, C₂-C₈ alkoxyalkyl, C₃-C₆ cycloalkyl, C₄-C₁₂ cycloalkylalkyl, aryl (aryl is phenyl or naphthyl optionally substituted with 1 to 3 substituents independently selected at each occurrence from C₁-C₄ alkyl, halo, cyano, —OR⁷, SH, —S(O)ₙR¹², —CO₂R⁸, —NR⁸COR⁷, —NR⁸CONR⁶R⁷, —NR⁸CO₂R¹², and —NR⁶R⁷), aryl(C₁-C₄ alkyl)-, heteroaryl or heteroaryl(C₁-C₄ alkyl)-, or —NR⁶R⁷.

2. The compound of claim 1, wherein X is N.

3. The compound of claim 1, wherein Y is O.

4. The compound of claim 1, wherein Z is CH₂.

5. The compound of claim 1, wherein R¹ is CH₃.

6. The compound of claim 1, wherein R² is H at each occurrence thereof.

7. The compound of claim 1, wherein R³ is H at each occurrence thereof.

8. The compound of claim 1, wherein R⁴ is C₂H₅, C₄H₉, C₅H₁₁, CH(C₂H₅)C₂H₅, CH₂-C₃ cyclopropyl or —CH₂—C₆H₅.

9. The compound of claim 9, wherein Ar is 2-bromo-4-isopropyl phenyl.

10. The compound of claim 1, wherein X is N, Y is O, Z is CH₂, R¹ is CH₃, R₂ and R₃ are each H at each occurrence thereof, Ar is 2-bromo-4-isopropyl phenyl and R⁴ is C₂H₅, C₄H₉, C₅H₁₁, CH(C₂H₅)C₂H₅, CH₂—C₃ cyclopropyl or —CH₂—C₆H₅.

11. A pharmaceutically acceptable salt form of the compound of claim 1.

12. A pharmaceutical composition comprising a pharmaceutically acceptable carrier and a therapeutically effective amount of a compound of claim 1.

13. A method of treating a mammal afflicted with a disorder characterized by excessive CRF expression which comprises administering to the mammal the pharmaceutical composition of claim 12.

14. The method of claim 13, wherein the disorder is anxiety, depression or affective disorder.
